(19) United States
(12) Patent Application Publication
(10) Pub. No.: US 2023/0029422 A1
(43) Pub. Date: Jan. 26, 2023

(54) METHOD AND SYSTEM FOR IN SITU TISSUE EXPANSION

(71) Applicant: Marz Medical, Inc., Mountain View, CA (US)

(72) Inventor: Joshua KORMAN, Los Altos Hills, CA (US)

(21) Appl. No.: 17/958,942

(22) Filed: Oct. 3, 2022

Related U.S. Application Data
(63) Continuation of application No. PCT/US21/26032, filed on Apr. 6, 2021.
(60) Provisional application No. 63/006,396, filed on Apr. 7, 2020.

(51) Int. Cl. A61B 90/00; A61B 90/90; A61F 2/12

(52) U.S. Cl. CPC A61B 90/02; A61B 90/90; A61F 2/12; A61B 2090/064

(57) ABSTRACT

A tissue expansion system includes an expandable bladder for placement beneath the skin corresponding to a tissue to be expanded, a fluid source, a pump coupled to both the fluid source and the expandable bladder to deliver inflation medium thereto, a controller for the pump, a pressure sensor for monitoring pressure within the expandable bladder and informing the action of the controller, and a carrier for holding at least the fluid source, pump, and controller. The carrier is wearable as a bra or form-fitting vest and is shaped to mimic the appearance of natural breasts for the patient. The fluid source can have an outward facing surface that is relatively rigid to maintain a curved shape mimicking that of natural breast(s). The inward facing surface of the fluid source may be flexible to conform to the shape of the underlying skin and tissue as it is expanded.

### CROSS-REFERENCE

This application is a continuation of PCT Application No. PCT/US21/26032, filed Apr. 6, 2021; which claims the benefit of U.S. Provisional Application No. 63/006,396, filed Apr. 7, 2020, which are incorporated by reference herein in their entirety.

The subject matter of this application is related to that of U.S. Pat. Application No. 13/592,138, filed Aug. 22, 2020 and granted as U.S. Pat. No. 9,265,921, 14/997,235, filed Jan. 15, 2016 and granted as U.S. Pat. No. 9,814,528, 15/730,293, filed Oct. 11, 2017, 16/807,668, filed Mar. 3, 2020, 14/468,908, filed Aug. 26, 2014 and now issued as U.S. Pat. No. 9,393,390, and 15/147,481, filed May 5, 2016, the full disclosures of which are incorporated herein by reference.

### BACKGROUND

1. Field of the Disclosure. The present disclosure relates generally to medical methods and devices. More particularly, the present invention relates to methods for expanding tissue using systems which monitor inflation pressure of an implanted expandable bladder.

Tissue defects in the skin and other tissues occur from a variety of causes including surgery, burns, traumatic injury, and congenital deformities. Such defects are often characterized by tissue “deficits” where there is insufficient skin or tissue present to cover or fill the affected body region in a normal or desired profile or pattern.

Tissue deficit may be treated by stimulating skin and/or tissue growth in the region of the defect. For example, “tissue expanders” may be implanted beneath a region of skin or within a volume of tissue which suffer from the deficit. By gradually inflating or otherwise expanding such tissue expander, the growth of skin and/or tissue can be promoted.

Presently, most tissue expanders are in the form of an implantable balloon with a valve that allows a physician to periodically inflate the balloon to increase its volume over successive office visits. As the patient may typically visit the doctor only about once per week, such periodic inflations often require relatively large volumes of inflation medium which can cause not only patient discomfort, but also tissue ischemia, concavities to underlying structures such as bone, and induce encapsulation of the implant causing capsular contraction and stiffening tissue around the expander.

In order to address such shortcomings, a number of “continuously” expanding devices have been proposed. For example, in U.S. Pat. Publication 2010/0010531, a device is described which allows the patient to periodically trigger a gas source within the implanted expander. Allowing the patient to control expansion, however, has its own drawbacks, and the patient may seldom follow an optimum inflation protocol to achieve the desired tissue expansion. Moreover, the use of a gas as the inflation medium is also disadvantageous.

For these reasons, it would be desirable to provide improved and alternative tissue expansion devices. In particular, it would be desirable to provide such devices which continuously and automatically deliver an inflation medium to an implanted expander over time in a more optimal and controlled pattern. It would be further desirable if such expanders and their supporting systems were adapted for patient convenience and comfort to further promote their use. At least some of these objectives may be met by the inventions described here and below.

2. Description of the Background Art. Relevant patents and publications include U.S. Pat. Nos. 6,668,836; 6,432,081; 5,549,672; 5,496,368; 5,005,591; 5,092,348; 4,955,905; and U.S. Pat. Publications 2011/152913; 2010/010531; 2008/051822; and 2004/147953. See also Logan and Hayden (1989) ISA, Paper #89-0207, pp. 27-33.

### SUMMARY

The present invention provides methods and systems which provide continuous, automated tissue expansion of a patient’s skin or other tissue. By “continuous” it is meant that the device may periodically deliver an inflation medium to an implanted expandable bladder in response to a monitored value, typically pressure within the inflatable bladder. By “automated” it is meant that the inflation of the tissue bladder or other expander may be initiated by the system itself, not by intervention from a physician or other personnel. Usually, as described below, the systems of the present invention may include an automated controller for monitoring the pressure or other patient value and for controlling a pump or other inflation medium delivery mechanism. Typically, the systems may maintain the pressure in the device at a level below 35 mmHg (in a range from 5 mmHg to 35 mmHg), usually below 20 mmHg, and most often at a level which does not exceed 5 -10 mmHg.

The tissue expansion component of the systems of the present disclosure may typically be an expandable bladder of the type which can be inflated with an inflation medium. The inflation medium is usually an “incompressible” medium, typically being a liquid, usually being saline or other biocompatible liquid medium. The bladder may have an expandable wall, usually being formed from an elastic material, such as silicone rubber or elastomer or the like. In other cases, the bladder could be at least partially formed from an inelastic or non-distensible material, such as a variety of inelastic polymers. In all cases, however, the expandable bladder may be configured to allow for controlled expansion. In the case of inelastic materials, the bladders may typically be pleated, folded, rolled or otherwise configured to allow unfurling during deployment. In general, the present invention can be used with any known or yet to be developed expandable device of the type intended to be implanted for tissue expansion.

The present disclosure specifically provides for monitoring of the pressure within the inflatable bladder or other tissue expander. Usually, the pressure may be monitored by a pressure sensor in an external portion of an inflation medium supply, i.e., one of the components which is not implanted. In other cases, however, it would be possible to deploy a pressure sensor within the inflatable bladder itself and/or a portion of an inflation medium feed tube connected to the bladder. Suitable pressure sensors include conventional piezoelectric transducers of the type which are conventionally used for pressure monitoring. The pressure may typically be monitored continuously in real time, but in another instance the pressure need only be monitored periodically at intervals separated by discrete time periods. Usually, however, the pressure may be monitored at least once each hour, typically being monitored much more often if not continuously.

The incompressible or other inflation fluid may be introduced into the expandable bladder whenever the monitored pressure falls below a lower threshold level, typically in the range from 0 mm Hg to 30 mm Hg. The lower threshold level may typically be held constant throughout an individual treatment, but could sometimes be changed at different times during the treatment protocol.

The introduction of incompressible inflation fluid may be terminated after a predetermined endpoint has been reached. The endpoint may typically occur when a higher pressure threshold has been reached within the expandable bladder typically in the range from 40 mm Hg to 50 mm Hg. The upper range, however, can be reduced when the integrity of the skin is compromised, for example when the skin has been previously compromised. Usually, the difference between the upper and lower pressure thresholds is at least 10 mm Hg, sometimes being as much as 20 mm Hg or more. In this way, expandable bladder can be inflated to a maximum pressure selected to effectively expand or distend the skin and tissue while causing minimum discomfort and reducing any side effects from such expansion. Only after the pressure returns to near a base level is additional fluid introduced to again raise the pressure to a level at or near a determined maximum value.

While relying on upper and lower pressure thresholds may usually be the preferred method for tissue expansion, in alternative embodiments a predetermined volume of fluid may be introduced whenever the pressure falls to or below the lower value described above. Such a predetermined volume of fluid may typically be in the range from 0.5 cc to 10 cc, usually from 3 cc to 5 cc, where the volume may be selected to provide effective tissue expansion with minimum risk of patient discomfort and trauma to the tissue. Still further alternatively, it would be possible to deliver a continuous flow or liquid fluid at a relatively low rate, e.g., 0.5 ml/hr to 3 ml/hr, for periods of hours or days, depending on the flow rates.

The methods of the present disclosure may find use whenever it is desirable to expand skin or other tissue surface, for example, following breast reconstructive surgery (mastectomies), when expanding tissue to be used for covering burn tissue or other defects, and the like. In a particular embodiment, the expandable bladder may be implanted in a subpectoral pocket following a mastectomy with or without an acellular dermal matrix. In such cases, the expandable bladder may be initially inflated with a small volume of saline or other inflation medium, typically in the range from 50 cc to 100 cc. In alternative embodiments, the expandable bladder may be implanted beneath skin or other tissue adjacent to a tissue defect. In such cases, the bladder may be inflated to expand the skin or tissue, and the expanded tissue may create a “flap” that can be used to cover the defect. When used following mastectomies, the bladder may optionally be left in place to provide the “breast implant”. Typically, the inflation tube as described below may be removed and the inflation chord in the bladder permanently sealed. In other procedures, the implant may typically be removed.

The present disclosure provides systems for expanding tissue and performing the methods as described above. The systems of the present invention comprise an expandable bladder adapted to be located beneath the region of the skin or other tissue to be expanded. The systems further comprise a pump adapted to be connected to a source of inflation medium, typically saline or other non-compressible medium, in order to deliver the inflation medium to the expandable bladder. Pumps suitable for use include positive displacement pumps such as reciprocating pumps (e.g., with a piston, plunger, and/or diaphragm, such as a syringe-like pump) and rotary pumps (e.g., with single or multiple rotors, such as a peristaltic pump). A pressure sensor adapted to monitor pressure within the expandable bladder (either directly or indirectly) on a substantially continuous basis is connected to a controller which receives such pressure data. The controller controls the pump to deliver inflation medium to the bladder whenever the pressure falls below a lower threshold value.

The systems of the present disclosure may further comprise a portable carrier configured to hold one or more of the pump, pressure sensor, or controller. The portable carrier may be in a form adapted to be worn by a patient, such as a bra-like jacket, vest, backpack, belt, or the like. A bra-like jacket may be shaped to mimic natural breasts. Alternatively, the carrier may be adapted with a handle or other means for allowing the patient to carry the carrier along with her or him. An exemplary system may comprise the source of inflation medium, for example, a bag of saline. In some embodiments, the source of inflation medium may be housed within the portable carrier. For example, the portable carrier may comprise a pouch for holding the source of inflation medium. The pouch may be located on a lateral side of the portable carrier. In some embodiments, the source of inflation medium is at least partially shaped to mimic the shape of one or more natural breasts. In some embodiments, the portable carrier may further comprise a pouch for the controller. This controller pouch may be located on a lateral side of the portable carrier, and the controller pouch may carry the pump and pressure sensor as well.

The controller may typically be programmed to deliver the inflation medium until a monitored pressure in the implant pressure reaches an upper threshold value, where the lower and upper threshold values are within the ranges set forth above. Alternatively, the controller may be programmed to deliver preselected inflation volumes after the lower threshold value has been reached and regardless of the higher pressure which is eventually achieved. The controller may typically be further programmed to stop delivering inflation medium entirely after a target total volume of the inflation medium has been delivered to the patient. In the case of post-mastectomy treatment, the total inflation medium delivered to the inflatable bladder may typically be in the range from 150 cc to 800 cc, more typically from 200 cc to 600 cc.

In another aspect of the present disclosure, a method for expanding tissue comprises locating an expandable bladder underneath a region of skin, subcutaneous tissue, or muscle to be expanded by introducing saline or another incompressible inflation medium. Preselected, incremental volumes of the incompressible inflation medium are delivered into the inflatable bladder at spaced-apart time intervals where delivery of one incremental volume is completed and delivery of a subsequent preselected incremental volume is commenced after a time delay, typically between 10 minutes and 3 hours, usually from 30 minutes to 2 hours, and typically about once an hour. By making the incremental volumes small, typically from 1 cc to 10 cc, and spacing the incremental inflation as just noted, the tissue expansion occurs at a slow, controlled rate and the tissue is allowed to yield or remodel between active expansion steps. As an additional protection against over stressing the tissue being expanded, pressure of the pumped inflation medium is monitored while each preselected incremental volume of inflation medium is being pumped into the bladder. Pumping is stopped if the monitored pressure exceeds an upper threshold level, typically in the range from 40 mmHg to 50 mmHg. Alternatively or in combination, pumping of the incremental volume can be terminated when the preselected incremental volume has been delivered to the bladder. If the pumping was stopped because the monitored pressure exceeded the upper threshold level, pumping the incremental volume may be recommenced when the monitored pressure falls back below a lower threshold level, typically in the range from 25 mmHg to 35 mmHg, and pumping of that incremental volume may continue until the preselected incremental volume has been reached or the monitored pressure once again exceeds the upper threshold level. The steps above can be repeated if the monitored pressure once again exceeds the upper threshold level before the preselected incremental volume has been delivered, and delivery of a plurality of subsequent incremental volumes can then begin at the preselected time intervals set forth above until a preselected cumulative volume of the inflation medium has been delivered to the expandable bladder. In the exemplary embodiments, the time required for delivery of each incremental volume of inflation medium may typically be only a small fraction of the time interval between successive incremental deliveries. For example, if the time interval between successive deliveries is on the order of one hour, the time necessary to driver one incremental volume may usually be under one minute. Thus, even if the high pressure threshold is exceeded more than once and pumping is interrupted, there may almost always be sufficient time to complete the delivery of each incremental volume during the time period between delivery of successive incremental volumes.

Usually, the successive incremental volumes and the time intervals between successive deliveries may be the same and within the ranges set forth above. Alternatively, either or both of the successive incremental volumes and the time intervals between successive deliveries may vary and be different at different times in the inflation delivery protocol.

In specific embodiments, locating the expandable bladder may comprise placing the expandable bladder beneath skin in a subcutaneous, subfascial or submuscular plane, located beneath a region of skin or tissue to be expanded. For example, this may be in a subpectoral pocket following mastectomy. The expandable bladder may usually be initially inflated with a volume of saline, typically in the range from 50 cc to 100 cc.

In other embodiments, the expandable bladder may be placed beneath skin, fascia or muscle located adjacent to a defect which, for example may be a burn scar. The expandable bladder may usually be initially inflated with a volume of saline, typically in the range from 20 cc to 100 cc. In these cases, the bladder may be removed after the skin has been expanded to create a flap and the flap is used to cover the defect.

In all these cases, the pumping may be performed with a constant speed positive displacement pump so that the predetermined incremental volume is provided by a predetermined incremental run time for the pump, and the preselected cumulative volume of the inflation medium is provided by a preselected cumulative run time of the pump.

In another aspect of the present disclosure, a system for expanding tissue comprises an expandable bladder adapted to be located beneath a region of skin to be expanded. A pump (e.g., a syringe or peristaltic pump) is configured to draw inflation medium from a source and to deliver said inflation medium to the expandable bladder. A pressure sensor is adapted to monitor pressure of the inflation medium being delivered by the syringe pump, and a delivery line connects the syringe pump to the inflatable bladder. A first one-way valve in the delivery line allows fluid from the syringe pump to flow to the inflatable bladder but blocks reverse flow from the bladder to the syringe. A refill line connects the pump to the source, and a second one-way valve in the refill line allows fluid flow from the source to the syringe and blocks reverse flow from the syringe to the source. A controller operates the pump to deliver inflation medium to the inflatable bladder and to draw inflation medium from the source, where said controller drives the pump periodically over a plurality of incremental time periods to deliver a plurality of incremental volumes of the incompressible inflation medium. The controller receives pressure data from the pressure sensor, and the controller stops the pump when a monitored pressure exceeds an upper threshold level and restarts the pump when the monitored pressure falls below a lower threshold value. The controller usually operates the pump for a predetermined incremental run time to deliver the incremental volume to the inflatable bladder.

In specific embodiments, the controller is programmed to deliver inflation medium until the pressure reaches an upper threshold value in the range from 40 mmHg to 50 mmHg, with pumping of the particular incremental volume being recommenced when the monitored pressure falls below the lower threshold level in the range from 25 mmHg to 35 mmHg. The preselected incremental volume of inflation medium is usually in the range from 1 cc to 10 cc, and the preselected time interval is in often the range from 10 minutes to 3 hours, more usually being in the range from 30 minutes to 2 hours, and typically being one hour.

In another aspect, disclosed herein, is a system for expanding tissue, said system comprising:
a) an expandable bladder adapted to be located beneath a region of skin to be expanded;
b) a pump adapted to be connected to a source of inflation medium and to deliver said inflation medium to the expandable bladder; and c) a portable carrier configured to hold one or more of the source of inflation medium and the pump, wherein the portable carrier is adapted to be worn by a patient.

In some embodiments, the portable carrier is configured to hold both the source of inflation medium and the pump. In some embodiments, the portable carrier comprises one or more pouches for holding the one or more of the source of inflation medium and the pump.

In some embodiments, the system further comprises: a) a pressure sensor adapted to monitor a pressure within the expandable bladder on a substantially continuous basis; and b) a controller which receives pressure data from the pressure sensor. In some embodiments, the controller is configured to control the pump to deliver the inflation medium to the expandable bladder when the pressure within the expandable bladder falls below a lower threshold value. In some embodiments, the controller is configured to control the pump to deliver the inflation medium to the expandable bladder until a predetermined volume of the inflation medium is delivered to the expandable bladder. In some embodiments, the controller is programmable. In some embodiments, the controller is configured to control the pump to deliver the inflation medium, terminate delivery of the inflation medium, and/or resume delivery of the inflation medium after a predetermined interval. In some embodiments, the predetermined interval is at least one hour. In some embodiments, the controller is programmed to deliver the inflation medium until the pressure within the expandable bladder reaches an upper threshold value. In some embodiments, the upper threshold level is in the range from about 5 mmHg to about 35 mmHg. In some embodiments, the controller is programmed to stop delivering the inflation medium after a target total volume of inflation medium has been delivered to the patient. In some embodiments, the target total volume of inflation medium is in the range from about 150 cc to about 800 cc. In some embodiments, the target total volume of inflation medium is in the range from about 200 cc to about 600 cc. In some embodiments, the portable carrier comprises a pouch for holding the controller. In some embodiments, the pouch is located on a lateral side of the portable carrier. In some embodiments, the controller is configured to receive an activation device for activating operation of the pump. In some embodiments, the activation device comprises a subscriber identification module (SIM) card. In some embodiments, the activation device is configured to enable operation of the pump for a prescribed duration after being received by the controller. In some embodiments, the prescribed duration is about 1 day, about 2 days, about 1 week, about 2 weeks, about 1 month, or about 3 months. In some embodiments, the pump and the controller are disposed with the same housing.

In some embodiments, the pump comprises a syringe pump. In some embodiments, the pump comprises a peristaltic pump.

In some embodiments, the portable carrier comprises a bra-like jacket, vest, a backpack, or a belt. In some embodiments, the bra-like jacket is shaped to mimic natural breasts.

In some embodiments, the expandable bladder is configured to be placed in a subpectoral pocket of the patient following a mastectomy. In some embodiments, the expandable bladder is initially inflated with a volume of saline in the range from about 50 cc to about 100 cc.

In some embodiments, the inflation medium comprises saline. In some embodiments, the system further comprises the source of the inflation medium. In some embodiments, the source of the inflation medium is housed within the portable carrier. In some embodiments, the source of inflation medium is at least partially shaped to mimic the shape of one or more natural breasts. In some embodiments, the portable carrier comprises a pouch for holding the source of inflation medium. In some embodiments, the pouch is located on a lateral side of the portable carrier.

In some embodiments, the system further comprises an activation device for activating operation of the pump. In some embodiments, the activation device comprises a subscriber identification module (SIM) card. In some embodiments, the activation device is configured to enable operation of the pump for a prescribed duration after being received by the controller. In some embodiments, the prescribed duration is about 1 day, about 2 days, about 1 week, about 2 weeks, about 1 month, or about 3 months.

The controller in preferred systems may be programmed or otherwise configured to pump inflation medium into the bladder and to terminate pumping of the incremental volume when said the incremental volume has been delivered to the bladder or when said the monitored pressure exceeds the upper threshold level. If the pumping was stopped because the monitored pressure exceeded the upper threshold level, the controller may recommence pumping of the incremental volume when the monitored pressure falls back below the lower threshold level and may continue pumping until the entire incremental volume has been reached or the monitored pressure once again exceeds the upper threshold level. The controller may repeat the stopping and recommencing if the monitored pressure once again exceeds the upper threshold level before the entire incremental volume has been delivered. After completing the delivery of each successive incremental volume, the controller may initiate delivery of the next successive incremental volume of inflation medium at a preselected time intervals until a preselected cumulative volume of the inflation medium has been delivered to the expandable bladder.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure may be obtained by reference to the following detailed description that sets forth illustrative embodiments, in which the principles of the present disclosure are utilized, and the accompanying drawings of which:

FIG. 1 is a block diagram illustrating the system components of the systems of the present disclosure.

FIG. 2 illustrates a vest adapted to carry certain system components used with an implantable inflatable bladder according to the present disclosure.

FIG. 3 is a block flow diagram illustrating the system components of an exemplary system constructed in accordance with the principles of the present disclosure.

FIG. 4 is a perspective, partially disassembled view of a driver assembly which forms a portion of the exemplary system of FIG. 3.

FIGS. 5A and 5B are front and rear views of the driver assembly of FIG. 3.

FIGS. 6A and 6B are detailed views taken along the line 6A-6A and line 6B-6B of FIG. 5B.

FIG. 7 is an exploded view of the exemplary system of the present disclosure showing the individual system components.

FIG. 8 is a logic diagram illustrating an exemplary operation protocol for the systems of the present disclosure.

FIG. 9A illustrates an embodiment of a wearable device adapted to carry components of the system used with an implantable inflatable bladder according to the present disclosure.

FIG. 9B is a schematic diagram of the device of FIG. 9A.

FIG. 10 illustrates another embodiment of a wearable device adapted to carry components of the system used with an implantable inflatable bladder according to the present disclosure.

FIG. 11A illustrates an exemplary front depiction of the device of FIG. 10 being worn by a user.

FIG. 11B illustrates an exemplary side depiction of the device of FIG. 10 being worn by a user.

FIG. 12A illustrates an exemplary depiction of a pump and controller housing for an embodiment disclosed herein.

FIG. 12B illustrates a top view of the pump and controller housing from FIG. 12A.

### DETAILED DESCRIPTION

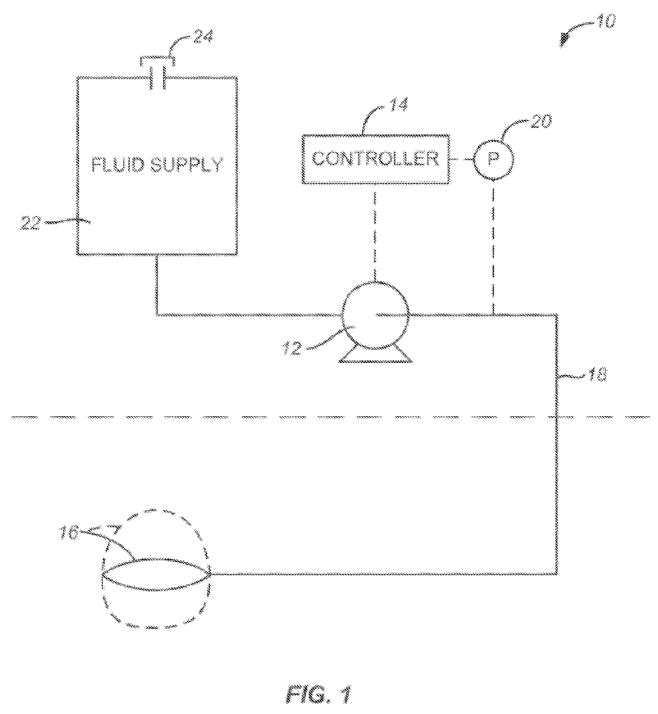

Referring to FIG. 1, a system 10 constructed in accordance with the principles of the present disclosure comprises a pump 12, a controller 14, and an inflatable bladder 16 connected to the pump by connecting tube 18. A pressure sensor 20 is connected to the connecting tube 18 (directly or indirectly) so that it may sense a static pressure within the inflation bladder 16. The pressure sensor is also connected to the controller 14 to provide a continuous realtime or periodic reading of the pressure in the bladder to the controller. The controller 14 typically comprises a digital microprocessor which has been programmed by conventional means to control the pump 12 as described above. A fluid supply 22, typically comprising an incompressible liquid such as saline, is attached to an input port of pump 12 so that it may be, in turn, be pumped to the inflatable bladder 16. The fluid supply typically has a port 24 to allow refilling of the supply with the desired fluid. Most portions of these system 10 may be maintained externally to the patient, as shown above the broken line in FIG. 1, while the inflatable bladder 16 and a portion of the connecting tube 18 may be implanted in the patient (below broken line). Typically, the connecting tube 18 may be transcutaneously placed through the patient’s skin and have a connecting end adapted for removable connection to the pump. Alternatively, the inflatable bladder may be connected through a connection port which lies substantially at the patient’s skin just beyond the transcutaneous insertion point. The corresponding portion of the tube may then be connected and disconnected from the pump as desired. Further inflation controllers that may be used with the system 10 are described in U.S. Pat. Application Nos. 15/147,481, filed on May 5, 2016 and 14/468,908, filed Aug. 26, 2014 and now issued as U.S. Pat. No. 9,393,390 on Jul. 19, 2016, the full disclosures of which are incorporated herein by reference.

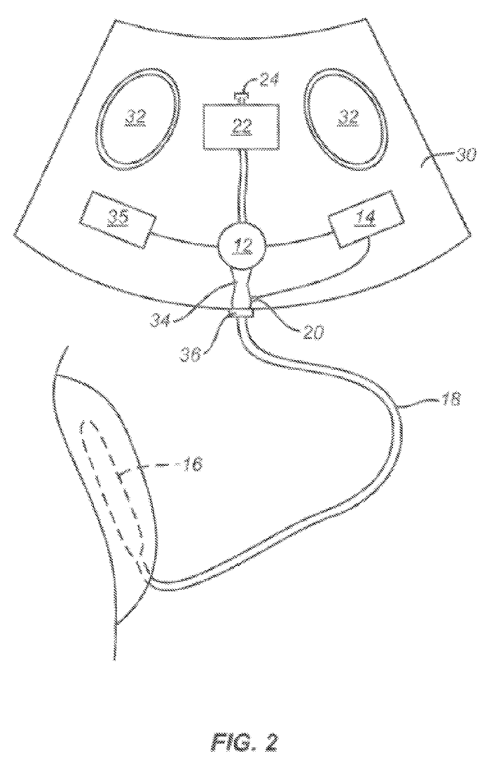

As illustrated in FIG. 2, the external system components of the system 10 may be mounted on a patient-wearable support, such as vest 30. The vest 30 may have the general layout of a vest garment including armholes 32, and may be further provided with the system components mounted on an interior and/or exterior surface thereof. As shown in FIG. 2, the pump 12 includes a short connecting tube 34 which extends to a connecting port 36 which may be removably attached to the connecting tube 18. The pressure sensor 20 may be located on the tube 34. Typically, a battery 35 or other power supply may also be provided to power the pump and controller.

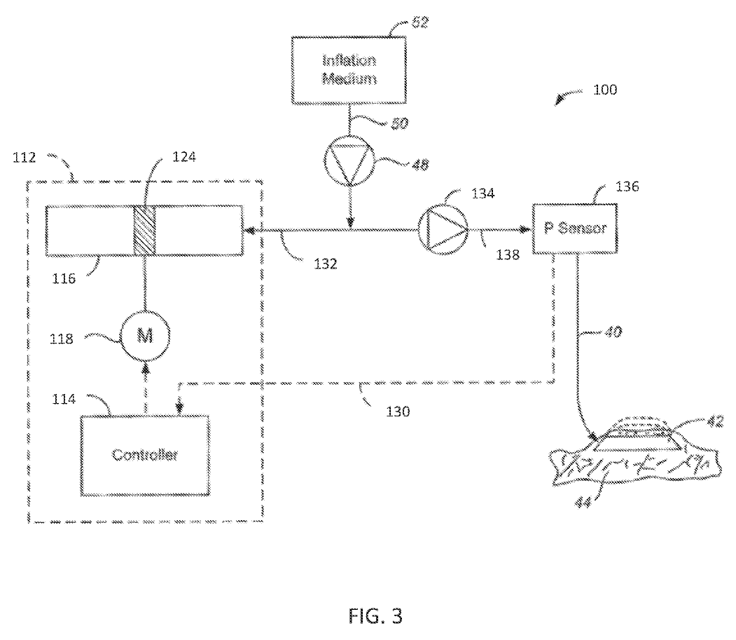

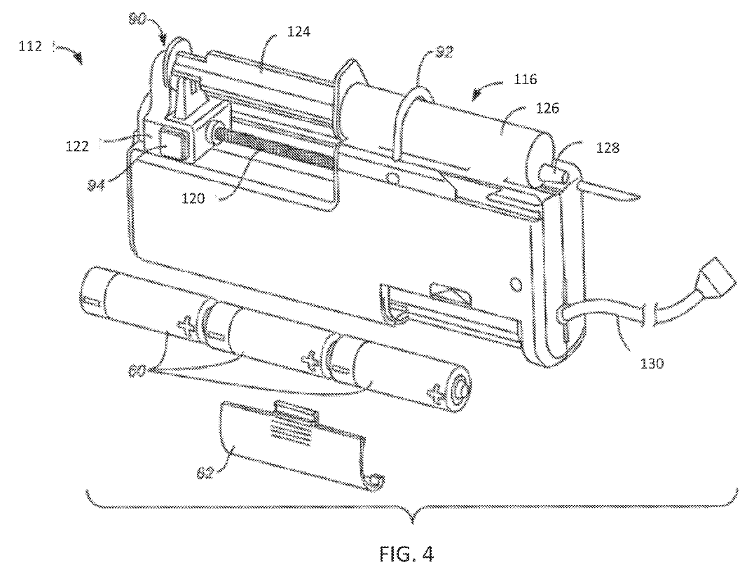

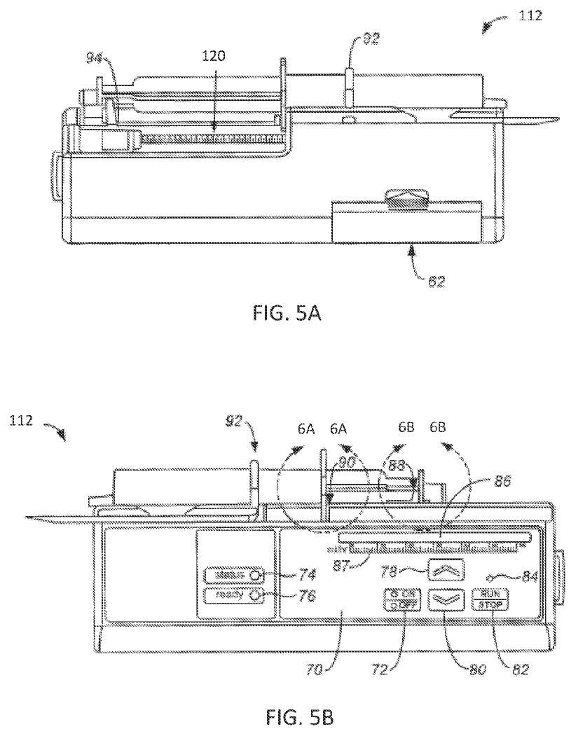

Referring in particular to FIG. 3, an exemplary system 100 constructed in accordance with the principles of the present disclosure comprises a driver assembly 112 which includes a controller 114, a syringe 116, and a motor 118. Typically, the motor may drive a lead screw 120 as shown in FIGS. 4 and 5A in order to advance a carriage 122 which is coupled to a plunger 124 of the syringe. The plunger 124, in turn, may either be advanced in a distal direction (to the right in FIG. 4) in order to deliver inflation medium from a barrel 126 of the syringe or may be retracted in a proximal direction in order to draw new inflation medium into the barrel, as will be described in more detail below. While a syringe or a piston or plunger-based pump is described, other pumps such as rotary and peristaltic pumps may also be suitable for use.

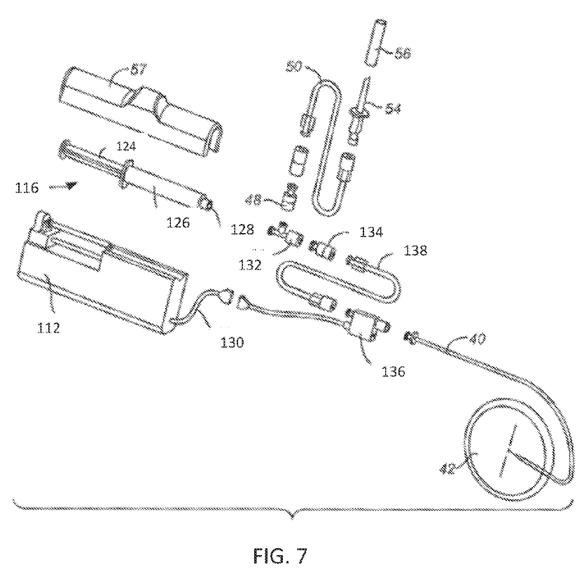

Referring now to FIGS. 3, 4, and 7, a connector 128 at a distal end of the syringe barrel 26 may be removably connected to a Y-fitting 132 which has two ports which are connected to a first one-way valve 134 and a second one-way valve 48, respectively. The first one-way valve 134, in turn, is removably connected to a connecting tube 138 which connects, at its distal end, to a pressure sensor 136. Pressure sensor 136, in turn, is connected to cable 130 which provides a pressure signal to the controller 114 within the delivery assembly 112. The pressure sensor 136 is further removably connected to a catheter 40 which in turn delivers inflation medium to the expandable and inflatable bladder 42. The first one-way valve 134 is oriented so that it allows flow from the syringe 116, which is caused by advancement of the plunger 124, to pass through the fitting 132, through the fitting 134, to the tube 138, through the pressure sensor 136, and finally through the catheter 40 into the inflatable bladder. The one-way valve 134 may prevent any backflow from the bladder 42 or elsewhere back into the syringe 116, thus reducing the risk of contamination.

The second one-way valve 48, in contrast, is oriented to cause inflation medium from a refill source 52 connected by a connecting tube 50 to flow back into the syringe barrel 126 when the plunger is retracted in order to draw the medium into the syringe. A needle 54 which is connectable to the tubing 50 is configured to be inserted into the source of saline or other inflation medium 52 so that the syringe maybe refilled between successive activations to deliver the inflation medium to the inflatable bladder 42. The controller 114 may be programmed to allow convenient refilling performed by the user. A cover 56 is provided to maintain sterility of the needle between successive uses. Another cover 57 is provided to cover and maintain sterility of the pump during use.

FIGS. 5A and 5B illustrate certain control and other features present on the driver assembly 112 of the systems of the present invention. A battery cover 62 can be removed to replace batteries as shown also in FIG. 4. A touch screen or other display 70 may be provided on the enclosure of the driver as a user interface. The display 70 may include, either virtually or mechanically, a power switch 72, a status light 74, a ready light 76, an incrementing key 78, a decrementing key 80, a run/stop key 82, and a syringe operation indicator 84.

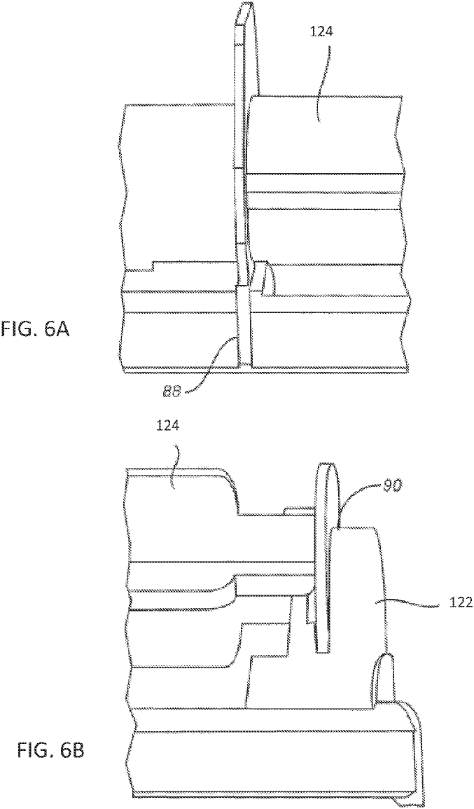

The pump maybe removed and replaced on the top of the driver assembly 112. In particular, the pump is held in place by a securing strap 92. In the case of a syringe pump, the plunger 124 is engaged by a slot 90 in the carriage 122, as best seen in FIG. 6B. Similarly, the syringe barrel flange 88 is held in another slot 88, as best seen in FIG. 6A. The position of the plunger maybe monitored, when the top 57 is in place, with a travel indicator 86 on the display panel 70. The carriage 122 maybe released from the lead screw 120 by a carriage release button 94.

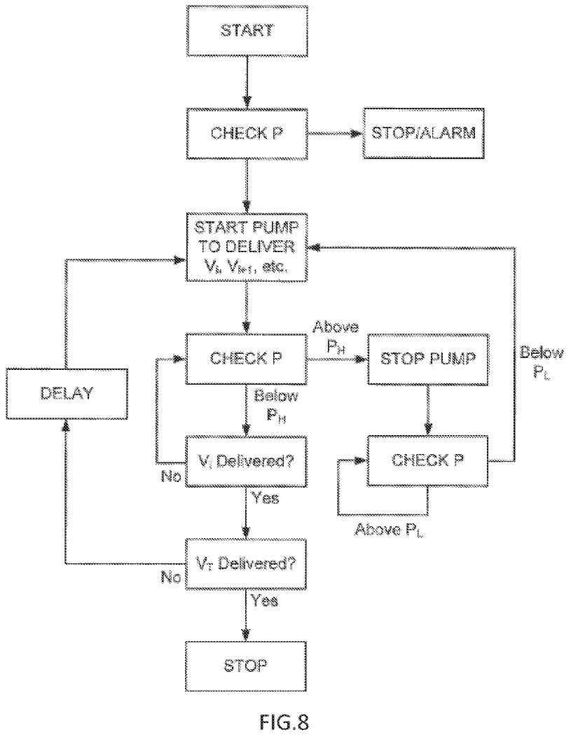

Referring now to FIG. 8, the inflation control system 100 of the present disclosure may be used by first connecting the catheter 40 to the pressure sensor 36 which in turn has been coupled to the driver 112, as described above. After making sure that the syringe 100 has a sufficient volume of inflation medium, the drive 112 may be turned on using switch 82 which may initiate the sequence of operations illustrated in FIG. 8. Initially, the pressure from sensor 36 is checked. If the pressure exceeds the high pressure threshold level PH, the driver may be stopped and an alert or alarm is raised since high pressure should not be present at this point in the protocol. Assuming that the initial pressure check is successful, the controller 114 may initiate power to the motor 118 in order to rotate the lead screw 120 which advances the carriage 122 which in turn drives the plunger 124 at a relatively low rate, typically the volumetric flow ranges set forth about. The pressure sensor 136 may monitor pressure while the syringe is delivering fluid, and the controller 114 may allow continued delivery for so long as the pressure does not exceed the high threshold value PH, again typically within the ranges set forth above. Assuming that no high pressure is detected, the syringe may be driven for a time sufficient to transfer the desired incremental volume Vi to the bladder 42. Typically, the volume may be from about 1 cc to 10 cc and it may take from 5 seconds to 60 seconds to complete the delivery.

If, however, the pressure sensor detects a pressure above the high threshold level PH, the controller may stop the pump and continue to monitor the pressure. For so long as the pressure remains above a low pressure threshold PL, which is typically 5 to 10 mmHg lower than the high pressure threshold PH, the pump may remain stopped. As soon as the pressure falls below this lower pressure threshold PL, the pump may be restarted and the inflation medium may continue to be delivered for so long as the pressure remains below the high pressure threshold PH. Pumping may continue until the entire incremental volume Vi has been delivered, at which time the pumping is stopped and not restarted until after the passage of a preselected time interval until the scheduled delivery of the successive incremental volumes Vi+1, i+2, ... , typically in the time ranges set forth above. Such successive incremental volume deliveries may continue until the total cumulative volume VT of inflation medium has been delivered to the inflatable bladder. Such total volume may take days or even weeks. In some instances, it may be desirable to divide the delivery of the total volume of inflation medium into stages, for example 2, 3, 4, 5, or even more stages, where the time between successive stages is greater than the normal time between the delivery of the incremental volumes.

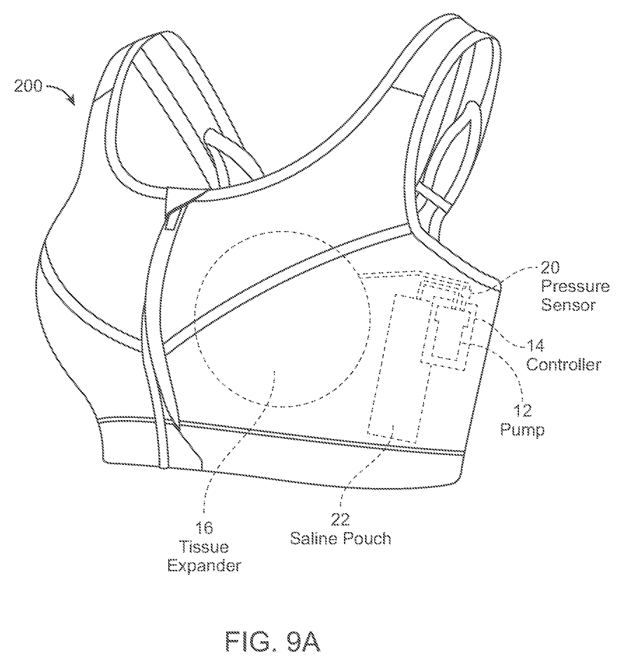

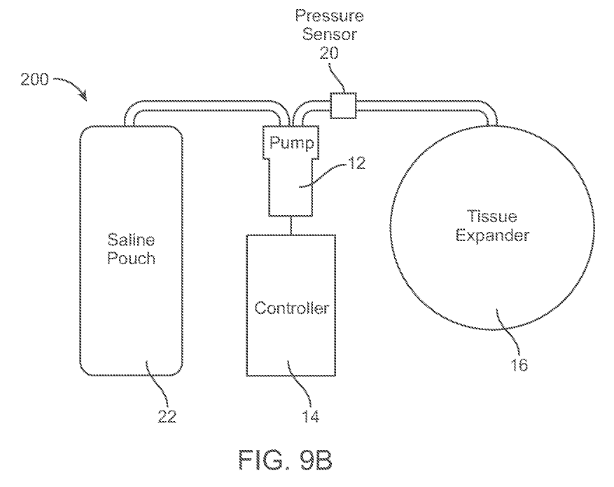

FIGS. 9A-9B depict an illustration of an exemplary embodiment of a patient-wearable device, such as a (sports) bra-like jacket 200, wherein the external system components of the system 10 may be mounted on . The bra-like jacket 200 may have the general layout of a women’s sports bra and may further include the system components mounted on an interior surface thereof. The bra-like jacket may be similar in configuration to the vest 30 and include any combination of the system components therewith, such as the pump 12, the controller 14, the tissue expander or inflatable bladder 16, the connecting tube 18, the pressure sensor 20, the fluid supply 22 (e.g., a saline pouch), the port 24, armholes 32, the short connecting tube 34, the battery 35, and/or the connecting port 36. The bra-like jacket 200 may be configured to give the wearer the appearance of breasts to a casual observer, particularly when worn as an undergarment. The bra-like jacket 200 may be made of materials such as Spandex, Nylon, cotton, polyester, a combination thereof, or the like. The bra-like jacket 200 may contain pockets or other accommodations for holding the controller/pump, the container or pouch of inflation fluid or saline (e.g., 250 cc of saline), a controller board, the pressure sensor, tubing from the container to the pump, tubing from the pump to the tissue expander, and/or valves as appropriate for detaching the tissue expander from the pump controller, for example, when the wearer wants to take a shower, change clothing, or otherwise desires to take off the bra-like jacket 200. The controller board may comprise an electronic box containing a circuit board, pump, housing, battery, and other components.

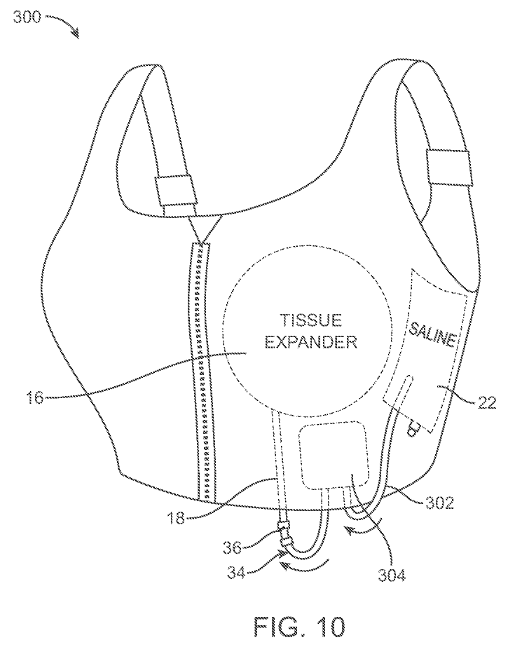

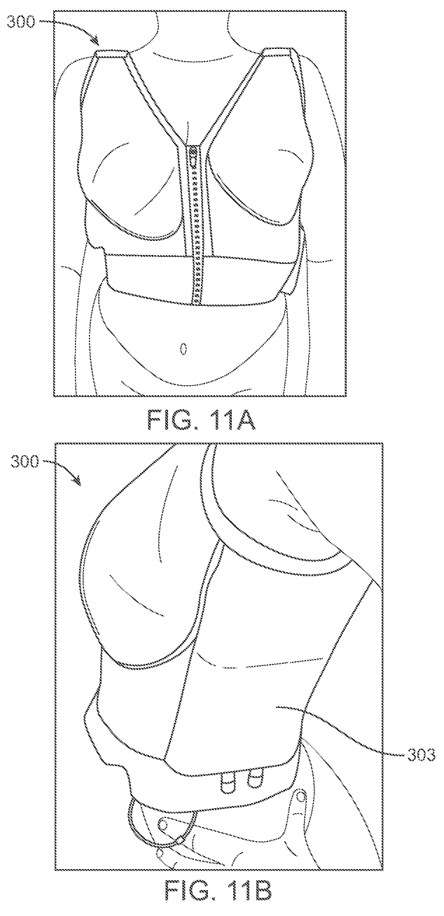

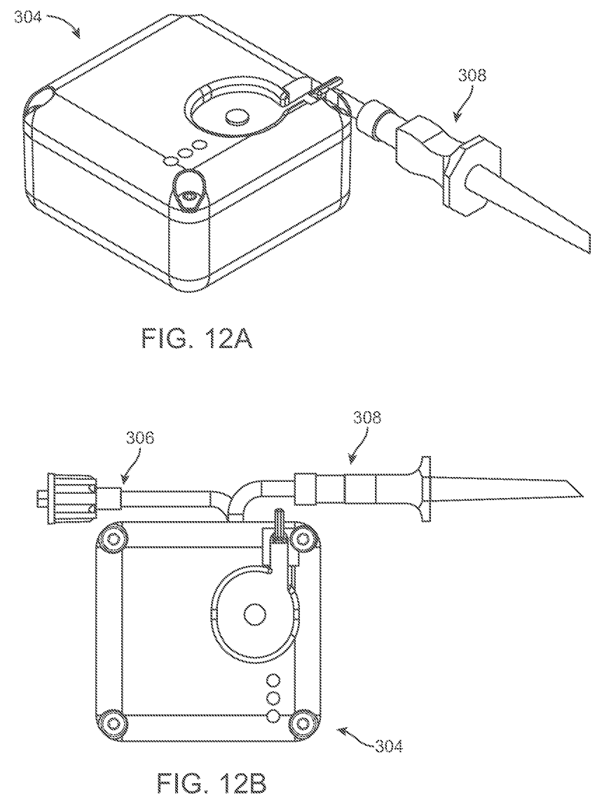

FIGS. 10 and 11A-B depict an illustration of another exemplary embodiment of a patient-wearable device, such as a (sports) bra-like jacket 300, wherein the external system components of the system 10 may be mounted on. The bra-like jacket 300 may have the general layout of a women’s sports bra and may further include the system components mounted on an interior surface thereof. The bra-like jacket may be similar in configuration to the vest 30 and include any combination of the system components therewith, such as the pump 12, the controller 14, the tissue expander or inflatable bladder 16, the connecting tube 18, the pressure sensor 20, the fluid supply 22 (e.g., a saline pouch), the port 24, armholes 32, the short connecting tube 34, the battery 35, the connecting port 36, and/or a supply connecting tube 302 between the fluid supply 22 and the pump. The pump 12 may be disposed with the controller 14 in the same housing 304. The connecting tube 18 may connect to the tissue expander via a luer lock. The tissue expander end may comprise a sealed needleless connecter. The pump and controller housing 304 end may comprise an exposed male luer lock when disconnected. The tubing 302 between fluid supply 22 and the housing 304 (e.g., pump, controller) may comprise a needleless connection to said fluid supply 22. The tube 302 may be in fluid communication with fluid supply using a spike (e.g., see inlet 308 in FIGS. 12A-12B).

FIGS. 12A-12B depict an exemplary embodiment of a pump and controller housing as described herein. The pump may include an inlet (e.g., 308), to receive fluid from the fluid supply 22, and an outlet (e.g., 306), to deliver fluid to the tissue expander. The pump and controller housing 304 may comprise the pump 12, a circuit board (e.g., as part of the controller 14) for controlling the pump flow rate (e.g., based on the pressure sensor), a battery, and/or other components (as known in the art). The battery may be the same battery as referred to in the vest 30, or a different battery. As aforementioned, the pump may comprise a peristaltic pump or a syringe pump. The pump and controller housing 304 may have a length of about 1“ to about 3”, and a width of about 1“ to about 3”. The bra-like jacket may comprise a button on the pump / controller housing 304, or elsewhere on the system components, that is configured to pause operation of the pump.

The pump may be configured to be activated by using an activation device that is received by the circuit board of the controller, and/or received by the pump. The activation device may be a subscriber identification module (SIM) card or the like. Removing the activation device may prevent the pump from operating. A given activation device (e.g., SIM card) may be configured to be valid for a specified duration before becoming invalid, at which point the pump will be prevented from operating until the invalid activation device is replaced with a valid activation device. By limiting the duration of a activation device, use of the system for expanding tissue, as described herein, can be restricted so as to help prevent a user from continuously and indefinitely using said system. The activation device may be prescribed to be valid for about 1 day, about 1 week, about 2 weeks, about 1 month, or about 3 months after being first used with a pump and controller configuration (as described herein). The activation device may be prescribed to be valid for any duration from about 6 hours to about 6 months.

The bra-like jacket 300 may be configured to give the wearer the appearance of breasts to a casual observer, particularly when worn as an undergarment (e.g., see FIGS. 11A-11B). The bra-like jacket 300 may be made of materials such as Spandex, Nylon, cotton, polyester, a combination thereof, or the like. The bra-like jacket 300 may contain pockets or other accommodations for holding the controller/pump, the fluid supply (e.g., see FIG. 11B, reference character 303), a controller board, the pressure sensor, tubing from the fluid supply to the pump, tubing from the pump to the tissue expander, and/or valves as appropriate for detaching the tissue expander from the pump / controller, for example, when the wearer wants to take a shower, change clothing, or otherwise desires to take off the bra-like jacket 300. The fluid supply may be inserted and removed from a pocket of the bra-like jacket 300 via a Velcro® flap from the top of a pocket. The fluid supply may be a container or pouch of inflation fluid or saline. The container or pouch may be configured to hold about 100 cc, about 250 cc, about 500 cc of inflation fluid or saline. The container or pouch may weigh about 0.5 pound, about 1 pound, about 1.2 pounds, or about 1.5 pounds. The pump and controller housing may weight about 50 grams, about 80 grams, or about 100 grams. The controller board may comprise an electronic box containing a circuit board, pump, housing, battery, and other components.

For any wearable support described herein (e.g., FIG. 9A, FIG. 10), the expandable bladder (also referred to herein as a tissue expander) may be implanted in a subpectoral or subcutaneous pocket as described herein. In some cases, the inflatable bladder may be implanted submuscular, partially submuscular, or subcutaneously (with or without acellular dermal matrix, or a synthetic mesh) as described herein. The inflatable bladder implant may be connected to a controller, pump, pressure sensor, and fluid supply (e.g., a saline pouch) as described herein. For example, FIGS. 9A-9B shows the bra-like jacket 200 with a pump 12, a controller 14 coupled to the pump 12 for control thereof, a tissue expander 16 fluidically coupled to the pump 12, and a source of inflation medium or saline container or bag or pouch (e.g., fluid supply 22) also fluidically coupled to the pump 12. Similarly, FIG. 10 shows the bra-like jacket 300 with a pump 12 and controller 14 disposed with a housing 304, a tissue expander 16 fluidically coupled to the pump 12, and a source of inflation medium or saline container or bag or pouch (e.g., fluid supply 22) also fluidically coupled to the pump 12 The system components are depicted in dashed lines to represent their location as being beneath an exterior surface of the bra-like jacket 200 / 300 (e.g., within the bra-like jacket, on an interior surface of the bra-like jacket, and/or subcutaneous, such as the tissue expander). As described above, for any wearable device described herein (e.g., FIG. 9A, FIG. 10) the pump may be a reciprocating and/or rotary pump, such as a syringe pump or a peristaltic pump. For example, the pumps may be such as those available from Binaca Pumps of Temecula, CA. The pressure sensor 20 may be adapted to monitor pressure within the expandable bladder 16 (either directly or indirectly) on a substantially continuous basis as described herein. The pressure sensor 20 may be connected to the controller 14 which receives such pressure data. The controller 14 may be adapted for substantially continuous operation in response to pressure within the tissue expander as described herein. The controller 14 may thus be able to deliver saline to the tissue expander 16 when the pressure within the tissue expander 16 falls below a predetermined threshold as described herein, while also pausing delivery of the fluid when the pressure is above a higher set threshold or reaches a predetermined maximum volume as described herein. For example, a pressure threshold may be any pressure from about 5 mmHg to about 40 mmHg. The predetermined maximum volume may be any volume between about 150 cc to about 800 cc, including any volume from about 200 cc to about 600 cc. The pump may be able to deliver the fluid (e.g., inflation medium, saline) at a flow rate of about 0.5 ml/hr to about 3 ml/hr.

The components of the system external to the patient may be configured to sit within the bra-like jacket 200, 300 and mimic the appearance of a normal, healthy breast (or breasts) during use. For instance, the outward facing surface of the fluid source or container may be shaped to mimic the appearance of a normal, healthy breast, and the inward facing surface may be adapted to conform to the changing shape of the patient. As the fluid is moved from the fluid source 22 to the tissue expander 16, the tissue may expand and fill in the space within the bra-like jacket which was previously occupied by the fluid source. The fluid source 22 may incrementally and substantially continuously reduce in size as the tissue expander incrementally and substantially continuously expands the tissue. As such, the appearance of the bra-like system may remain substantially the same (e.g., as breasts) during the whole tissue expansion process. The outward facing surface of the fluid source or container 22 may be more rigid than the inward facing surface so as to maintain the external appearance and shape of the bra-like system as the system conforms to the expanded tissue of the patient. After a pre-determined maximum volume has been delivered to the tissue expander, for example over a period of 102 weeks, the tissue expander may be removed and a suitable permanent breast implant may be added to the expanded tissue pocket. By providing a system which can mimic the feel of a bra and look of a breast, patients may be better able to and/or be more comfortable performing routine, daily tasks without a feeling of loss (e.g., missing breast(s) or self-consciousness as the tissue expansion takes place).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for expanding tissue, said system comprising:
(a) an expandable bladder adapted to be located beneath a region of skin to be expanded;
(b) a pump adapted to be connected to a source of inflation medium and to deliver said inflation medium to the expandable bladder; and
(c) a portable carrier configured to hold one or more of the source of inflation medium and the pump, wherein the portable carrier is adapted to be worn by a patient.

2. A system as in claim 1, wherein the portable carrier is configured to hold both the source of inflation medium and the pump.

3. A system as in claim 1, wherein the portable carrier comprises one or more pouches for holding the one or more of the source of inflation medium and the pump.

4. A system as in claim 1, further comprising:
(a) a pressure sensor adapted to monitor a pressure within the expandable bladder on a substantially continuous basis; and
(b) a controller which receives pressure data from the pressure sensor.

5. A system as in claim 4, wherein the controller is configured to control the pump to deliver the inflation medium to the expandable bladder when the pressure within the expandable bladder falls below a lower threshold value.

6. A system as in claim 4, wherein the controller is configured to control the pump to deliver the inflation medium to the expandable bladder until a predetermined volume of the inflation medium is delivered to the expandable bladder.

7. A system as in claim 4, wherein the controller is programmable.

8. A system as in claim 4, wherein the controller is configured to control the pump to deliver the inflation medium, terminate delivery of the inflation medium, and/or resume delivery of the inflation medium after a predetermined interval.

9. A system as in claim 8, wherein the predetermined interval is at least one hour.

10. A system as in claim 4, wherein the controller is programmed to deliver the inflation medium until the pressure within the expandable bladder reaches an upper threshold value.

11. A system as in claim 10, wherein the upper threshold level is in the range from about 5 mmHg to about 35 mmHg.

12. A system as in claim 4, wherein the controller is programmed to stop delivering the inflation medium after a target total volume of inflation medium has been delivered to the patient.

13. A system as in claim 12, wherein the target total volume of inflation medium is in the range from about 150 cc to about 800 cc.

14. A system as in claim 13, wherein the target total volume of inflation medium is in the range from about 200 cc to about 600 cc.

15. A system as in claim 4, wherein the portable carrier comprises a pouch for holding the controller.

16. A system as in claim 15, wherein the pouch is located on a lateral side of the portable carrier.

17. A system as in claim 4, wherein the controller is configured to receive an activation device for activating operation of the pump.

18. A system as in claim 17, wherein the activation device comprises a subscriber identification module (SIM) card.

19. A system as in claim 17, wherein the activation device is configured to enable operation of the pump for a prescribed duration after being received by the controller.

20. A system as in claim 19, wherein the prescribed duration is about 1 day, about 2 days, about 1 week, about 2 weeks, about 1 month, or about 3 months.

21. A system as in claim 4, wherein the pump and the controller are disposed with the same housing.

22. A system as in claim 1, wherein the pump comprises a syringe pump.

23. A system as in claim 1, wherein the pump comprises a peristaltic pump.

24. A system as in claim 1, wherein the portable carrier comprises a bra-like jacket, vest, a backpack, or a belt.

25. A system as in claim 24, wherein the bra-like jacket is shaped to mimic natural breasts.

26. A system as in claim 1, wherein the expandable bladder is configured to be placed in a subpectoral pocket of the patient following a mastectomy.

27. A system as in claim 26, wherein the expandable bladder is initially inflated with a volume of saline in the range from about 50 cc to about 100 cc.

28. A system as in claim 1, wherein the inflation medium comprises saline.

29. A system as in claim 1, further comprising the source of the inflation medium.

30. A system as in claim 29, wherein the source of the inflation medium is housed within the portable carrier.

31. A system as in claim 29, wherein the source of inflation medium is at least partially shaped to mimic the shape of one or more natural breasts.

32. A system as in claim 29, wherein the portable carrier comprises a pouch for holding the source of inflation medium.

33. A system as in claim 32, wherein the pouch is located on a lateral side of the portable carrier.

34. A system as in claim 1, further comprising an activation device for activating operation of the pump.

35. A system as in claim 34, wherein the activation device comprises a subscriber identification module (SIM) card.

36. A system as in claim 34, wherein the activation device is configured to enable operation of the pump for a prescribed duration after being received by the controller.

37. A system as in claim 36, wherein the prescribed duration is about 1 day, about 2 days, about 1 week, about 2 weeks, about 1 month, or about 3 months.
